(19) United States
(12) Patent Application Publication
(10) Pub. No.: US 2021/0393237 A1
(43) Pub. Date: Dec. 23, 2021

(54) ULTRASOUND DIAGNOSTIC APPARATUS

(71) Applicant: Konica Minolta, Inc., Tokyo (JP)

(72) Inventors: Tatsushi CHIHARA, Tokyo (JP); Yasuhiro NAKAMURA, Kanagawa (JP); Takahiko SHIRAISHI, Tokyo (JP)

(21) Appl. No.: 17/351,179

(22) Filed: Jun. 17, 2021

(30) Foreign Application Priority Data
Jun. 23, 2020 (JP) 2020-108063

(51) Int. Cl. A61B 8/00

(52) U.S. Cl. CPC A61B 8/4433; A61B 8/56; A61B 8/4427; A61B 8/54

(57) ABSTRACT

An ultrasound diagnostic apparatus includes: a power connector provided with a power supply terminal to which a power cable is connected; an apparatus body provided with a power connector connection section which includes a body-side terminal to be connected to the power supply terminal and to which the power connector is insertable and detachable; and an attraction section configured to attract facing surfaces of the power connector and the power connector connection section to each other.

### CROSS REFERENCE TO RELATED APPLICATIONS

The entire disclosure of Japanese Patent Application No. 2020-108063 filed on Jun. 23, 2020 is incorporated herein by reference in its entirety.

### BACKGROUND

### Technological Field

The present invention relates to an ultrasound diagnostic apparatus.

### Description of Related Art

In recent years, portable ultrasound diagnostic apparatuses have been developed such that users (e.g., physicians) can perform medical examinations or the like at homes of patients.

When such ultrasound diagnostic apparatus is used in a hospital or the like, it is possible to improve the operability when performing diagnostics or the like by, for example, connecting (docking) the apparatus to a cart-type structure (also simply referred to as “cart”) provided with a power source section, casters (wheels) and the like.

Further, by being connected (docked) to a power connector provided in the cart, or an optional cradle, the ultrasound diagnostic apparatus can perform the operation of each section by external power source, charging of an inner battery, and a communication with a host terminal, for example.

Here, a power cable to be connected to the ultrasound diagnostic apparatus is desirably fixed so as not to loosen or come off during movement and use of the apparatus (see, for example, Japanese Patent Application Laid-Open No. 2013-150716 (hereinafter, referred to as “PTL 1”)).

Unfortunately, for the conventional ultrasound diagnostic apparatuses, when the connector of the power cable is inserted and attached, cases where the connector is inserted incompletely have often occurred.

In such cases, since the ultrasound diagnostic apparatus operates by power of the internal power source (battery) instead of the external power source, there is a problem such as an occurrence of a shutdown operation during diagnostics due to a battery exhaustion.

To solve the above-described problem, the art disclosed in PTL 1 provides a configuration that prevents the incomplete insertion by using a tool, which involves, however, an another problem in that fixing and detaching the tool take time and effort.

### SUMMARY

An object of the present invention is to provide an ultrasound diagnostic apparatus capable of reducing time and effort in connecting a connector of a power cable and ensuring the effectiveness of the connection.

To achieve at least one of the abovementioned objects, according to an aspect of the present invention, an ultrasound diagnostic apparatus reflecting one aspect of the present invention includes:
a power connector provided with a power supply terminal to which a power cable is connected;
an apparatus body provided with a power connector connection section which includes a body-side terminal to be connected to the power supply terminal and to which the power connector is insertable and detachable; and
an attraction section configured to attract facing surfaces of the power connector and the power connector connection section to each other.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a perspective view of a specific exemplary ultrasound diagnostic system according to an embodiment of the present invention;

FIG. 2 is a perspective view of a body and the like of an ultrasound diagnostic apparatus in a state of being detached from the ultrasound diagnostic system in the state illustrated in FIG. 1;

FIG. 3 illustrates a schematic configuration of a power connector to be connected to a rear side of the apparatus body;

FIG. 4 is an enlarged perspective view of a lock latch extracted from the power connector;

FIG. 5 illustrates a more specific configuration of the power connector;

FIG. 6A is a transparent view of the power connector illustrated from a rear side; and

FIG. 6B is a transparent view of a specific exemplary plug to be inserted into the power connector.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an ultrasound diagnostic apparatus and its stand according to the present embodiment will be described in detail with reference to the accompanying drawings.

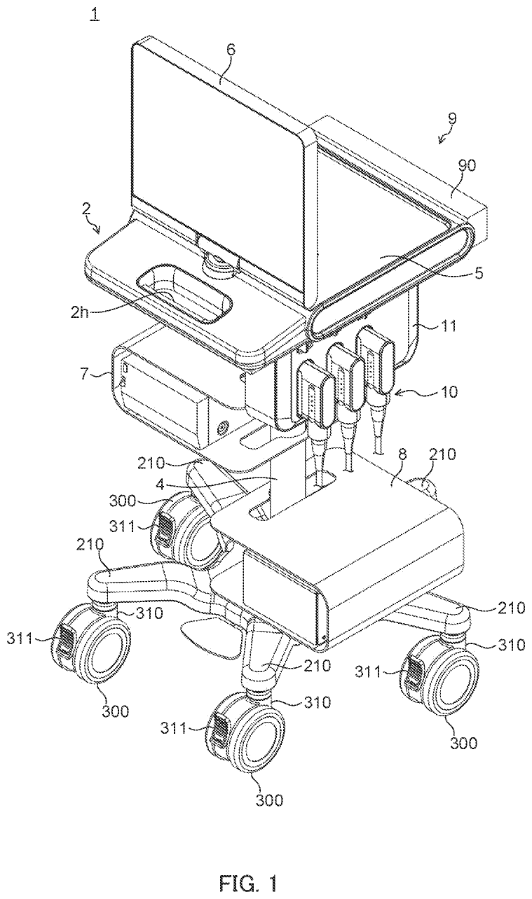

FIG. 1 is an external view of ultrasound diagnostic system 1 according to the present embodiment as viewed from the rear side of the system (assembly). Ultrasound diagnostic system 1 includes, for example, from the bottom, legs 210 movable on a floor by a plurality of (five in this example) casters 300, cylindrical lifter 4 supported by legs 210, and power source section 8, printer 7, and probe selector 11 which are assembled to lifter 4.

Ultrasound diagnostic system 1 also includes docking section 9 provided with docking member 90 and the like at a position above probe selector 11 along lifter 4.

Moreover, ultrasound diagnostic system 1 of the present embodiment has a configuration in which, with respect to docking section 9, casing 2 as a main body of an ultrasound diagnostic apparatus (hereinafter, simply referred to as “apparatus body”) is attachably and detachably connected (docked).

Hereinafter, a configuration of each section mentioned above will be described more specifically. As illustrated in FIG. 1, in ultrasound diagnostic system 1 according to the present embodiment, caster holding sections 310 are rotatably connected (shaft supported) to lower surfaces of legs 210, respectively, and casters 300 as wheels are rotatably held by caster holding sections 310, respectively.

Such ultrasound diagnostic system 1 has a so-called cart-type configuration and can move freely, for example, in a hospital room, by rotation of casters 300 and/or caster holding sections 310 on an installation surface (e.g., a floor surface).

That is, in a state where directions of casters 300 are aligned as illustrated in FIG. 1, when ultrasound diagnostic system 1 is pushed to a right upper side in the figure, casters 300 rotate without rotation of caster holding sections 310; and thus, the system can move with the overall apparatus.

By contrast, when ultrasound diagnostic system 1 is pushed to another side such as a right lower side in the figure, casters 300 rotate after shafts holding casters 300 (or phases of casters 300) rotate in the movement direction; and thus, the system can move with the overall apparatus.

Further, as illustrated in FIG. 1, caster holding sections 310 include rotation stoppers 311 that function as a brake or movement prevention. In a specific example, rotation stoppers 311 are set in predetermined positions (hereinafter referred to as “lock position”) by a sliding operation of a user or the like so as to be in contact with corresponding casters 300, and thereby prevent (lock) casters 300 from rotating.

In the present embodiment, when each of rotation stoppers 311 is set to the lock position, all (5 in this example) casters 300 in contact with the floor surface cannot rotate, which enables preventing or suppressing the overall ultrasound diagnostic system 1 from considerably moving (displacing) from the floor surface.

Accordingly, at the time of attaching and detaching apparatus body 2 to docking section 9 to be described later, respective rotation stoppers 311 are desirably also set in the lock position.

With reference to FIG. 1, lifter 4 is provided upright in a central position of ultrasound diagnostic system 1, and power source section 8, printer 7, probe selector 11, and docking section 9 are sequentially assembled from the bottom with respect to lifter 4. Moreover, apparatus body 2 is configured to be attachable to and detachable from docking section 9.

Power source section 8 for supplying power to each section of ultrasound diagnostic system 1 is provided above legs 210 with lifter 4. Power source section 8 includes a transformer or the like (not illustrated), converts, by using the transformer or the like, a current supplied from the indoor power outlet serving as an external power source (e.g., AC/DC and voltage conversion) and supplies the converted current to each section of ultrasound diagnostic system 1.

Printer 7 is provided above power source section 8 with lifter 4. Printer 7 has a function to print out, for example, imaging results of ultrasound images and diagnostic results from ultrasound diagnostic system 1.

In addition, probe selector 11 is provided above printer 7 with lifter 4. Probe selector 11 detachably holds a plurality of (three in the illustrated example) ultrasound probes 10.

Further, docking section 9 is provided at the top of lifter 4. Docking section 9 includes, for example, a substantially plate-shaped tray (not illustrated) for placing (mounting) apparatus body 2 and block-shaped docking member 90 that is integral with the tray.

Apparatus body 2 is a substantially box-shaped outer package and accommodates various components (e.g., processors, various interfaces, batteries) constituting the ultrasound diagnostic apparatus. In addition, as illustrated in FIG. 1, handle 2h to be held by the user is formed on one end of apparatus body 2.

Although not illustrated in detail, in a specific example, on an upper surface of apparatus body 2, operation input section 5 such as various input keys (hardware switches) and a trackball is provided, for receiving a user-operation. Further, between operation input section 5 of apparatus body 2 and above-described handle 2h, operation display section 6 such as a touch-input type liquid crystal display panel is turnably provided upright with respect to the upper surface of apparatus body 2 (see FIG.1).

The ultrasound diagnostic apparatus in apparatus body 2 is operated with a repeatedly chargeable/dischargeable inner battery or power supplied from power source section 8 and generates a drive signal for transmitting ultrasound waves to ultrasound probe 10. The generated drive signal is transmitted to corresponding ultrasound probe 10 through a signal line (not illustrated) connected to probe selector 11. The ultrasound diagnostic apparatus in apparatus body 2 also performs various kinds of electrical processing, such as generating an ultrasound image based on the signal received from ultrasound probe 10 through the signal line.

In a specific example, lifter 4 may be configured to be vertically telescopic (and/or extendable) and horizontally rotatable. In this case, the user can adjust by vertically telescoping (and/or extending) lifter 4, for example, operation input section 5 and operation display section 6 of apparatus body 2 to height positions for easier operation. Similarly, the user can adjust operation input section 5 or the like to a rotation position for easier operation, by horizontally rotating lifter 4.

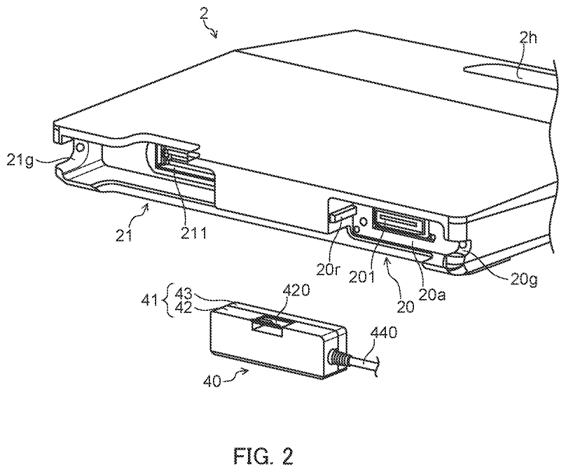

In addition, lifter 4 also has a function of passing the signal lines and various cables, that is, housing a power cable and a power connector extending from power source section 8 (see FIG. 2, as appropriate) and connecting them to docking section 9 through a predetermined opening (not illustrated in FIG. 1).

Thus, in ultrasound diagnostic system 1 of the present embodiment, the user (the doctor) can detach apparatus body 2 from docking section 9 and carry it with necessary ultrasound probe 10 or the like, and thereby performs imaging or medical examinations at, for example, the home of the patient.

In this case, the ultrasound diagnostic apparatus (apparatus body 2, ultrasound probe 10, and/or the like) is operated with the internal power source (battery), or a power cable which is an accessory may be used to supply power to the apparatus through the external power source of the patient's house.

Incidentally, as described above, the power cable to be connected to the ultrasound diagnostic apparatus (apparatus body 2) is desirably fixed so as not to loosen or come off during movement and use of the apparatus. Unfortunately, for the conventional ultrasound diagnostic apparatuses, when the connector of the power cable is inserted and attached, cases where the connector is inserted incompletely have often occurred.

In such cases, since the ultrasound diagnostic apparatus operates by power of the internal power source (battery) instead of the external power source, there is a problem such as an occurrence of a shutdown operation during diagnostics due to a battery exhaustion.

As described above, the art disclosed in PTL 1 for the above problems has a problem in that fixing and detaching the tool take time and effort.

In view of the circumstances described above, in the present embodiment, the following configuration is adopted in order to reduce time and effort in connecting the connector of the power cable and ensure the effectiveness of the connection.

That is, in the ultrasound diagnostic apparatus according to the present embodiment is configured to include an attraction section for attracting facing surfaces to each other of the power connector and the power connection section of apparatus body 2 to which the power connector is connected.

Desirably, force with which the attraction section attracts the facing surfaces to each other is greater than a pressing force required to insert the power connector into the power connector connection section of apparatus body 2.

In a specific example, the attraction section includes a magnet (first magnet) provided on one of facing surfaces of the power connector and the power connector connection section, and a magnetic material provided on the other of the facing surfaces, or a second magnet provided on the other of the facing surfaces and having a magnetic pole opposite to the first magnet.

The above-described magnets (first magnet and second magnet) are not particularly limited in terms of material, size, arrangement, or the like, and also may be an electromagnet as well as a permanent magnet.

Furthermore, in order to prevent, for example, coming-off caused by an unexpected external force or the like in a state where the connector of the power cable is connected, a coming-off prevention section for preventing the power connector from coming-off from the power connector connection section of apparatus body 2 may be provided.

In a specific example, the coming-off prevention section is configured to be locked to a corresponding part of the power connector connection section of apparatus body 2, and is configured to include an operation section for performing an operation of releasing the locking in such a locked state.

Hereinafter, a specific example of the above-mentioned configuration will be described with reference to FIG. 2 and following drawings.

FIG. 2 is a perspective view of apparatus body 2 and power connector 40, which are detached from the system state illustrated in FIG. 1. Note that, for convenience of description, a configuration on apparatus body 2 side (e.g., operation display section 6) is omitted in FIG. 2.

As illustrated in FIG. 2, at an end opposite to handle 2h in apparatus body 2, substantially recessed power connector connection section 20 to which power connector 40 is attached/detached, and probe connector connection section 21 to which a connector of above-described ultrasound probe 10 (not illustrated) is connected are provided.

Of the above, probe connector connection section 21 on a left side in FIG. 2 includes, for example, probe connector 211 in which a plurality of terminals to be electrically connected to the connector of ultrasound probe 10 are arranged, and groove 21g into which a cable of ultrasound probe 10 (not illustrated) is fitted.

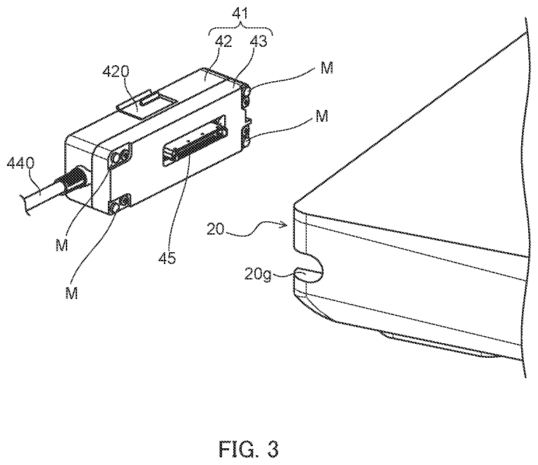

On the other hand, power connector connection section 20 on a right side in FIG. 2 includes, for example, connector (hereinafter referred to as “body side connector”) 201 provided with an electrode on a main body side (main body side terminal) to which electrode section 45 (see FIG. 3) provided with an electrode (power supply terminal) of power connector 40 is connected, and groove 20g into which power cable 440 of power connector 40 is fitted.

More specifically, in power connector connection section 20, abutment surface 20a to which casing 41 of power connector 40 is abutted is provided on an inner side of apparatus body 2, and rib-shaped projection 20r is provided along an attaching/detaching direction of power connector 40 from abutment surface 20a.

Of the above, projection 20r has a shape corresponding to a shape of a groove-shaped recess (not illustrated) provided on one side surface side corresponding to casing 41 of power connector 40 (i.e., a side opposite to a side where power cable 440 is pulled out), and thereby serves to regulate or guide a posture of power connector 40 at the time of attachment/detachment thereof.

Meanwhile, abutment surface 20a of power connector connection section 20 has an opening at its central area, and body side connector 201 is provided in the opening. In relation to the present invention, abutment surface 20a corresponds to a “facing surface” on one side, that is, a side of power connector connection section 20.

In the present embodiment, power connector 40 is connected to an outlet of a common external power source (e.g., a 100 to 120V AC power supply), and thereby serves to supply power of the external power source to apparatus body 2.

Power connector 40 includes, for example, an outlet plug to be plugged into/unplugged from the above-described outlet (not illustrated), power cable 440 connected to the outlet plug at one end thereof (hereinafter, simply referred to as cable 440), and casing 41 that accommodates the other end of cable 440 and is attached/detached to/from above-described power connector connection section 20 of apparatus body 2.

Casing 41 of power connector 40 is a substantially box-shaped member formed by combining two members of outer half 42 and inner half 43, and has an outer shape corresponding to an inner shape of power connector connection section 20 of apparatus body 2.

FIG. 3 is a perspective view of power connector 40 as viewed from the opposite side to that illustrated in FIG. 2. As illustrated in FIG. 3, outer half 42 of casing 41 of power connector 40 has a slightly longer depth than inner half 43. Moreover, substantially plate-shaped lock latch 420 is provided on an upper surface of outer half 42 of casing 41. Lock latch 420 corresponds to the “coming-off prevention section” of the present invention.

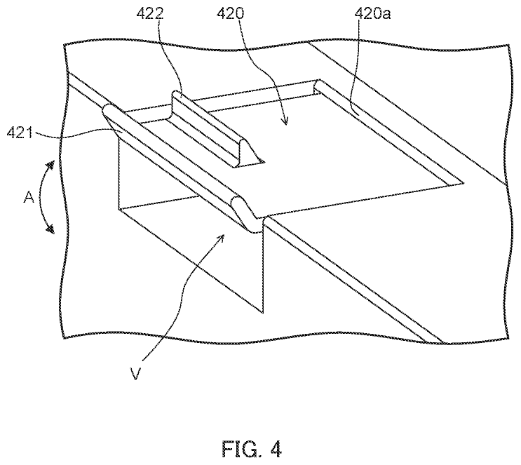

FIG. 4 is a notched perspective view of lock latch 420 illustrated in FIG. 3 in an enlarged manner. As referred to FIG. 4, lock latch 420 is configured to be turnable in two-headed arrow A direction in FIG. 4 around connection section 420a on one end thereof.

In a specific example, an energizing section such as a spring (e.g., leaf spring or coiled spring) for energizing lock latch 420 in contact with a lower surface of lock latch 420 is provided in space V where lock latch 420 can move inside.

Moreover, as illustrated in FIG. 4, an end opposite to connection section 420a of lock latch 420 is finger rest 421 projecting outward from two surfaces of outer half 42. Finger rest 421 bulges out to bend upward in FIG. 4 with respect to an upper surface of lock latch 420 such that the user can easily presses it.

Further, as illustrated in FIG. 4, on the upper surface of lock latch 420, lock claw 422 is formed upward and extending parallel to finger rest 421. Lock claw 422 has a major role of a function (coming-off preventing section) to suppress or prevent power connector 40 from coming off, due to the external force or the like, from power connector connection section 20, by being engaged (locked) with (to) a groove (not illustrated) formed in a corresponding upper inner surface of power connector connection section 20 of apparatus body 2.

Besides, above-described finger rest 421 corresponds to the “operation section” of the present invention and serves to cause the user to perform the operation of releasing the locking (i.e., pressing and moving operation) in a state where power connector 40 is locked to the groove of power connector connection section 20.

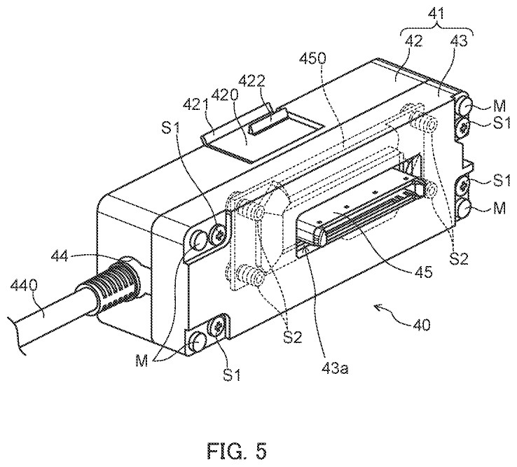

Next, a description will be given mainly of an internal configuration of casing 41 of power connector 40 with reference to FIG. 5 and the following drawings.

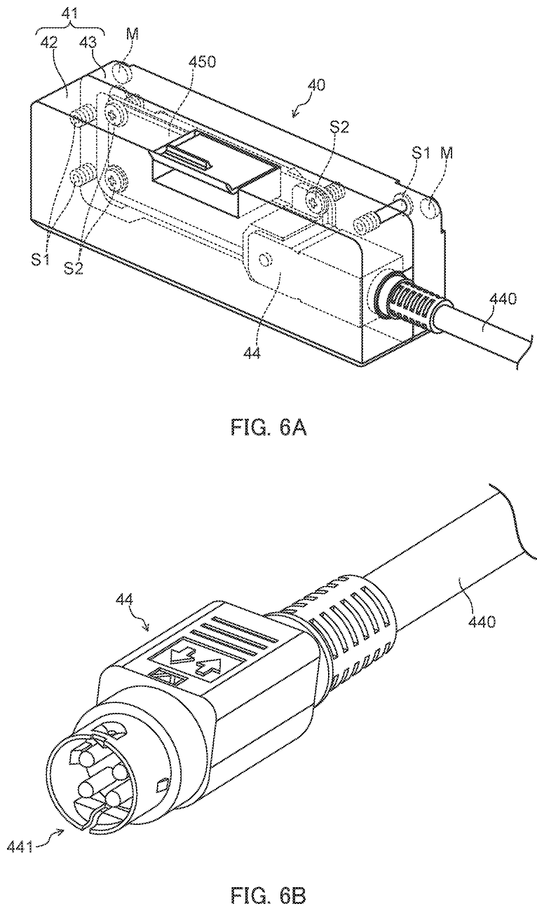

As referred to perspective views of FIGS. 5 and 6A, electrode section 45 of power connector 40 is placed so as to project outwardly from opening 43a of inner half 43. More specifically, electrode section 45 is a floating connector which is movable in a substantially central area of the first surface of substrate 450 housed within casing 41.

The floating connector includes a substantially flat tubular insulation member having a planner shape smaller than opening 43a of inner half 43 and a plurality of electrode terminals arranged on an inner surface of the insulation member, and is movably provided in the opening shape defined by opening 43a.

In the illustrated example, substrate 450 is fixed to an inner surface of inner half 43 by a plurality of (four in the illustrated example) screws S2 being screwed from the second surface of substrate 450. Meanwhile, electrode section 45 (floating connector) provided on the first surface side of substrate 450 projects so as to face outward from opening 43a of inner half 43, and is movably provided in vertical and horizontal directions with respect to opening 43a.

As referred to in FIG. 6A, the above-described end of cable 440 is housed inside of outer half 42 facing the second surface of substrate 450. In a specific example, a plurality of female-type terminals (not illustrated) is provided on the second surface side of substrate 450, and the end of cable 440 is attached/detached to/from the female-type terminals.

According to the above-described configuration, for example, as illustrated in FIG. 6B, general-purpose type (commercially available) cable 440 having plug 44 with a plurality of male-type terminals 441 may be used.

As referred to the transparent views of FIGS. 5 and 6A, in this example, outer half 42 and inner half 43 are fixed to each other by a plurality of (four in the illustrated example) screws 51 being screwed from an outer surface of inner half 43.

Further, in the present embodiment, on the outer surface of inner half 43, a plurality of (four in this example) magnets M is provided, which serves as a part of the “attraction sections” that is attracted to above-described power connector connection section 20 of apparatus body 2. In the illustrated example, magnet M is provided at each corner in the rectangular outer surface of inner half 43.

On the other hand, in power connector connection section 20 of apparatus body 2, magnetic materials such as iron plates serving as another part of the “attraction sections” are provided at positions corresponding to respective magnets M in abutment surface 20a to which the outer surface of inner half 43 is abutted.

As another configuration, a magnetic material such as an iron plate may be provided on the outer surface of inner half 43, and magnet M may be provided at the corresponding position of abutment surface 20a. Alternatively, magnets M may be provided on both the outer surface of inner half 43 and abutment surface 20a. Note that, when the configuration in which magnets M are attracted to each other is adopted, polarities (distinction between S-pole and N-pole) of magnets M facing each other are required to be different from each other.

In the present embodiment, a material (component member) constituting magnet M is not particularly limited, and any material such as a neodymium magnet and a ferrite magnet may be used.

Whereas, in the present embodiment, an attracting force (unit: Newton) by above-described magnets M, is desirably set to be greater than a pressing force required to insert (fit deeply and completely) power connector 40 into power connector connection section 20 of apparatus body 2.

To achieve such a force relationship, such as the material, area, and/or number of magnets M may be appropriately adjusted. In addition, the material, area, and/or number of magnets M may be adjusted such that friction that may occur between the inner surface of power connector connection section 20 and the outer surface of power connector 40 and friction that occurs between body side connector 201 and electrode section 45 of power connector 40 do not become excessively large.

According to the present embodiment with such a configuration, for example, when the user holds power connector 40 and inserts it into power connector connection section 20 of apparatus body 2, the respective parts are connected as follows.

That is, during the operation of inserting power connector 40 into power connector connection section 20 of apparatus body 2, electrode section 45 is electrically connected by being fitted into body side connector 201 while each magnet M of inner half 43 is attracted to the corresponding magnetic material of abutment surface 20a.

More particularly, when the user holds power connector 40 and inserts it into power connector connection section 20, lightly pushing power connector 40 alone causes the outer surface of power connector 40 to be attracted, by the attraction force of magnets M, to abutment surface 20a (each facing surface) of power connector connection section 20 of apparatus body 2, and thereby these facing surfaces are abutted to each other.

As a result, electrode section 45 of power connector 40 and body side connector 201 can be electrically and physically connected to each other, completely, which enables effectively preventing incomplete connections, such as a so-called “half-done insertion.”

Further, during an operation of deeply inserting power connector 40 into power connector connection section 20 of apparatus body 2, lock claw 422 of lock latch 420 of outer half 42 is locked to the groove of power connector connection section 20.

Consequently, a problem such as coming-off of power connector 40 from power connector connection section 20 of apparatus body 2 due to an unexpected external force or the like can be effectively prevented.

Furthermore, when detaching power connector 40 from apparatus body 2, the user simply has to pull lock latch 420 toward him/her, after placing a finger on finger rest 421 of lock latch 420 so that lock latch 420 moves downward. Thus, according to the present embodiment, power connector 40 can be detached with a simple operation.

As described above, the ultrasound diagnostic apparatus of the present embodiment is configured to include: power connector 40 provided with the power supply terminal (electrode section 45) to which power cable 440 is connected; apparatus body 2 provided with power connector connection section 20 which includes the body-side terminal (body side connector 201) to be connected to the power supply terminal (electrode section 45) and to which power connector 40 is insertable and detachable; and the attraction section (e.g., magnets M) for attracting facing surfaces of power connector 40 and power connector connection section 20 to each other. Thus, the apparatus is capable of reducing time and effort in connecting power cable 440 to apparatus body 2 and ensuring the effectiveness of the connection.

The embodiments described above are merely examples of specific implementation of the present invention, and the technical scope of the present invention should not be restrictively interpreted by these embodiments. That is, the present invention may be implemented in various forms without departing from the spirit thereof or the major features thereof.

Although embodiments of the present invention have been described and illustrated in detail, the disclosed embodiments are made for purpose of illustration and example only and not limitation. The scope of the present invention should be interpreted by terms of the appended claims.

What is claimed is:

1. An ultrasound diagnostic apparatus, comprising:
a power connector provided with a power supply terminal to which a power cable is connected;
an apparatus body provided with a power connector connection which includes a body-side terminal to be connected to the power supply terminal and to which the power connector is insertable and detachable; and
an attractor configured to attract facing surfaces of the power connector and the power connector connection to each other.

2. The ultrasound diagnostic apparatus according to claim 1, wherein
a force with which the attractor attracts the facing surfaces to each other is greater than a pressing force required to insert the power connector into the power connector connection.

3. The ultrasound diagnostic apparatus according to claim 1, wherein
the attractor includes: a first magnet provided on one of the facing surfaces of the power connector and the power connector connection; and a magnetic material or a second magnet provided on another one of the facing surfaces, the second magnet having a magnetic pole opposite to the first magnet.

4. The ultrasound diagnostic apparatus according to claim 1, wherein
the power connector includes a coming-off preventer configured to prevent the power connector from coming-off from the power connector connection by being locked to the power connector connection.

5. The ultrasound diagnostic apparatus according to claim 4, wherein
the coming-off preventer includes an operation section configured to release a locking in a state where the power connector is locked to the power connector connection.

6. The ultrasound diagnostic apparatus according to claim 1, wherein
the power connector accommodates a terminal of the power cable attachable to and detachable from the power supply terminal.

7. The ultrasound diagnostic apparatus according to claim 1, wherein
the power supply terminal is a floating connector movable with respect to a casing of the power connector.
